(19) United States
(12) Patent Application Publication
(10) Pub. No.: US 2024/0269449 A1
(43) Pub. Date: Aug. 15, 2024

(54) Silicone Low Profile Port with Rigid Baseplate and Stem

(71) Applicant: Bard Peripheral Vascular, Inc., Franklin Lakes, NJ (US)

(72) Inventors: Christian Andersen, Queen Creek, AZ (US); Bryon Ray Densley, Rochester, MN (US); Jessica Hoye, Phoenix, AZ (US); Ian N. Thomas, West Bountiful, UT (US); Diana Fiumefreddo, Salt Lake City, UT (US)

(21) Appl. No.: 18/561,627

(22) PCT Filed: May 20, 2021
(86) PCT No.: PCT/US21/33375

§ 371 Date: Nov. 16, 2023

(51) Int. Cl. A61M 39/02

(52) U.S. Cl. CPC A61M 39/0208; A61M 2039/0226; A61M 2039/0232; A61M 2205/0216; A61M 2205/04

(57) ABSTRACT

Embodiments disclosed herein are directed to a port system including a collapsible reservoir and associated methods thereof. The port can include a body defining a reservoir and formed of a compliant material. The port body can transition between an expanded configuration and a collapsed configuration. The port can further include a port stem or a base plate, formed of a rigid or needle impenetrable material. The base plate can be aligned with a floor of the reservoir to prevent an access needle from traversing the bottom surface of the reservoir. The port, or the reservoir, can transition to the collapsed configuration to provide a reduced overall size or outer profile for insertion and/or between access events. The port can require a smaller incision site, required fewer stitches or no stitches at all, improving patient recovery times, patient comfort, reducing scarring and improving aesthetics.

### SUMMARY

Briefly summarized, embodiments disclosed herein are directed to a port system including a collapsible reservoir and associated methods thereof. Where ports, or similar rigid medical devices, are placed subcutaneously, the size of the device, e.g. overall volume, transverse height, or the like, can cause surrounding tissues to stretch and erode. For example, where the port is placed on a chest wall, the port can protrude relative to the skin surface. The skin tissues stretched over the port can erode causing sores or even exposing the port. This is further exacerbated by abrasion from clothes or seat belts.

Embodiments disclosed herein are directed to a port system including a port having a body that defines a reservoir and formed of a compliant material. The port body can transition between an expanded configuration and a collapsed configuration. The port can further include a port stem or a base plate, formed of a rigid or needle impenetrable material. The base plate can be aligned with a floor of the reservoir to prevent an access needle from traversing the bottom surface of the reservoir. Advantageously, the port can transition to the collapsed configuration to provide a reduced overall size or outer profile for insertion, and/or between access events. The port can require a smaller incision site, requiring fewer stitches or no stitches at all to close the incision site, improving patient recovery times, patient comfort, reducing scarring, erosion, and improving aesthetics.

Disclosed herein is a subcutaneous access port including, a port stem formed of a first material, being a rigid material and having a first durometer, and a body defining a reservoir in fluid communication with the port stem, and formed of a second material, being a flexible material and having a second durometer, the body transitionable between an expanded configuration and a collapsed configuration, the collapsed configuration defining a smaller outer profile of the port.

In some embodiments, the first material includes one of a plastic, polymer, metal, alloy, or composite. In some embodiments, the second material includes one of a plastic, polymer, elastomer, synthetic rubber, organic rubber, silicone rubber, or composite. In some embodiments, the port body in the expanded configuration defines one of a first port height, a first port width, or a first port length, and wherein the port body in the collapsed configuration defines one of a second port height, a second port width, or a second port length. In some embodiments, one of the second port height is less than the first port height, the second port width is less than the first port width, or the second port length is less than the first port length.

In some embodiments, the port body in the expanded configuration defines a first port volume, and the port body in the collapsed configuration defines a second port volume, the second port volume being less than the first port volume. In some embodiments, the reservoir in the expanded configuration defines one of a first reservoir height, a first reservoir width, or a first reservoir length, and wherein the reservoir in the collapsed configuration defines one of a second reservoir height, a second reservoir width, or a second reservoir length. In some embodiments, one of the second reservoir height is less than the first reservoir height, the second reservoir width is less than the first reservoir width, or the second reservoir length is less than the first reservoir length. In some embodiments, the reservoir in the expanded configuration defines a first reservoir volume, and the reservoir in the collapsed configuration defines a second reservoir volume, the second volume being less than the first volume.

In some embodiments, the subcutaneous access port further includes a needle penetrable septum disposed over the reservoir and configured to provide percutaneous access thereto by a needle. In some embodiments, the needle penetrable septum is formed of either the second material or a silicone rubber. In some embodiments, the first durometer of the first material is larger than the second durometer of the second material. In some embodiments, the first material is a rigid material and is substantially resistant to flexible deformation, and wherein the second material is an elastically deformable material. In some embodiments, the body is elastically deformable from the expanded configuration to the collapsed configuration.

In some embodiments, the body is elastically deformable from the collapsed configuration to the expanded configuration. In some embodiments, the subcutaneous access port further includes a third material, being elastically deformable and including a third durometer greater than the second durometer and less than the first durometer. In some embodiments, the third material is disposed on an outer surface of the body. In some embodiments, the third material is disposed on a wall of the reservoir. In some embodiments, the subcutaneous access port further includes a base plate formed of one of the first material or a flexible needle impenetrable material. In some embodiments, the base plate and the stem are formed integrally as a single unitary piece.

Also disclosed is a method of placing a port subcutaneously including, providing a port including, a port stem configured to engage a catheter and provide fluid communication therewith, a body defining a reservoir and transitionable between an expanded configuration and a collapsed configuration, the body in the collapsed configuration defining a smaller overall volume than the body in the expanded configuration, and a needle penetrable septum disposed over the reservoir, transitioning the port from the expanded configuration to the collapsed configuration, inserting the port body through an insertion site to place the port subcutaneously, and transitioning the port from the collapsed configuration to the expanded configuration.

In some embodiments, the method further includes accessing the port percutaneously with a needle before transitioning the port from the collapsed configuration to the expanded configuration. In some embodiments, the method further includes accessing the port percutaneously with a needle after transitioning the port from the collapsed configuration to the expanded configuration. In some embodiments, the port stem is formed of a first material being a rigid material including one of a plastic, polymer, metal, alloy, or composite, and the body is formed of a second material being a flexible material includes one of a plastic, polymer, elastomer, synthetic rubber, organic rubber, silicone rubber, or composite. In some embodiments, the first material includes a first durometer and the second material includes a second durometer, the second durometer being less than the first durometer.

In some embodiments, the port in collapsed configuration defines a width that less than a width of the port in the expanded configuration, a height that less than a height of the port in the expanded configuration, or a length that less than a length of the port in the expanded configuration. In some embodiments, the reservoir of the port in collapsed configuration defines a width that less than a width of the reservoir in the expanded configuration, a height that less than a height of the reservoir in the expanded configuration, or a length that less than a length of the reservoir in the expanded configuration. In some embodiments, the reservoir in the collapsed configuration defines a volume that less than a volume of the reservoir in the expanded configuration.

In some embodiments, the needle penetrable septum is formed of the same material as the body. In some embodiments, the body further includes a third material displaying elastically deformable mechanical properties and including a third durometer less than the first durometer and greater than the second durometer. In some embodiments, the third material is disposed on an outside surface of the body. In some embodiments, the third material is disposed on a wall of the reservoir. In some embodiments, the method further includes a base plate formed of the first material, or a flexible needle impenetrable material. In some embodiments, the base plate and the port stem are formed integrally as a single unitary piece.

Also disclosed is a method of manufacturing an access port including, forming a port stem including a first material, being a rigid material and having a first durometer, and forming a body defining a reservoir in fluid communication with the port stem, and including a second material, being a flexible material and having a second durometer, the body transitionable between an expanded configuration and a collapsed configuration, the collapsed configuration defining a smaller outer profile of the port.

In some embodiments, the first material includes one of a plastic, polymer, metal, alloy, or composite. In some embodiments, the second material includes one of a plastic, polymer, elastomer, synthetic rubber, organic rubber, silicone rubber, or composite. In some embodiments, the port body in the expanded configuration defines one of a first port height, a first port width, or a first port length, and wherein the port body in the collapsed configuration defines one of a second port height, a second port width, or a second port length.

In some embodiments, one of the second port height is less than the first port height, the second port width is less than the first port width, or the second port length is less than the first port length. In some embodiments, the port body in the expanded configuration defines a first port volume, and the port body in the collapsed configuration defines a second port volume, the second port volume being less than the first port volume. In some embodiments, the reservoir in the expanded configuration defines one of a first reservoir height, a first reservoir width, or a first reservoir length, and wherein the reservoir in the collapsed configuration defines one of a second reservoir height, a second reservoir width, or a second reservoir length.

In some embodiments, one of the second reservoir height is less than the first reservoir height, the second reservoir width is less than the first reservoir width, or the second reservoir length is less than the first reservoir length. In some embodiments, the reservoir in the expanded configuration defines a first reservoir volume, and the reservoir in the collapsed configuration defines a second reservoir volume, the second volume being less than the first volume.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a port coupled to a catheter, in accordance with embodiments disclosed herein.

FIG. 2A shows a longitudinal cross-section view of a port, in accordance with embodiments disclosed herein.

FIG. 2B shows a longitudinal cross-section view of a port, in accordance with embodiments disclosed herein.

FIG. 2C shows a longitudinal cross-section view of a port, in accordance with embodiments disclosed herein.

FIG. 2D shows a plan, cross-section view of a port, in accordance with embodiments disclosed herein.

FIG. 3A shows a lateral, cross-section view of a port in an expanded configuration, in accordance with embodiments disclosed herein.

FIG. 3B shows a lateral, cross-section view of a port in a collapsed configuration, in accordance with embodiments disclosed herein.

FIG. 3C shows a longitudinal, cross-section view of a port in an expanded configuration, in accordance with embodiments disclosed herein.

FIG. 3D shows a longitudinal, cross-section view of a port in a collapsed configuration, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

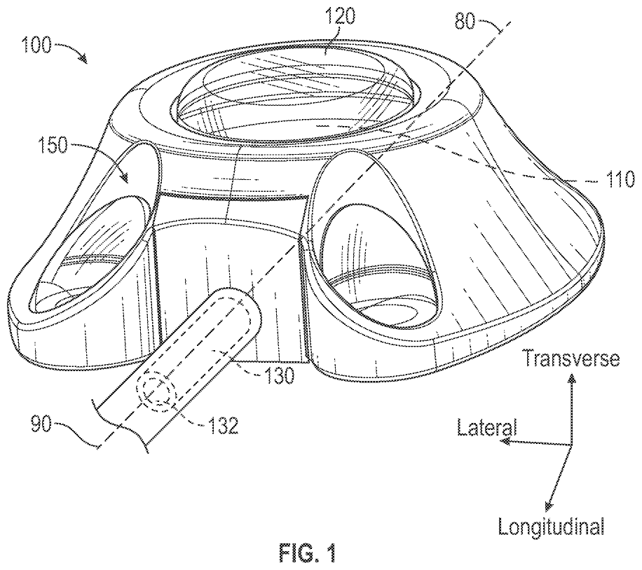

To assist in the description of embodiments described herein, as shown in FIG. 1, a longitudinal axis extends substantially parallel to an axial length of the catheter. A lateral axis extends normal to the longitudinal axis, and a transverse axis extends normal to both the longitudinal and lateral axes. As used herein, a horizontal plane extends along the lateral and longitudinal axes. A vertical plane extends normal to the horizontal plane.

Unless defined otherwise, all technical and scientific terms used herein have the same meaning as commonly understood by those of ordinary skill in the art.

FIG. 1 shows a vascular access device, or (“port”) 100 including a collapsible reservoir 110 configured to facilitate subcutaneous placement. The port 100 can generally include a port body 150 defining the reservoir 110 and include a needle penetrable septum 120 disposed thereover. In an embodiment, the septum 120 can be formed of a silicone rubber or similar suitable material. In an embodiment, the septum material can substantially define a Shore D 50 durometer, however greater or lesser Shore D durometers are also contemplated. In use, the reservoir can be accessed percutaneously by a needle penetrating the septum 120 and fluidly accessing the reservoir.

The port 100 can further include a port stem 130 extending along a stem axis 80 and defining a stem lumen 132 that is in fluid communication with the reservoir 110. In an embodiment, the stem axis 80 can extend substantially parallel to the longitudinal axis. The stem 130 can be configured to be coupled to a catheter 90 or similar device, configured to access a vasculature of a patient. The catheter 90 can include an elongate body defining a lumen extending therethrough. In an embodiment, the port stem 130 can be formed of a first material. In an embodiment, the first material can be a substantially rigid material, defining a first durometer, such as a plastic, polymer, polyoxymethylene (POM or “Delrin”), polyether ether ketone (PEEK), metal, alloy, stainless steel, titanium, composite, or the like. In an embodiment, the first material be formed of a resilient material. In an embodiment, the first material can resist any elastic or plastic deformation. In an embodiment, the first durometer can be equal to or greater than Shore D 70. However, other durometers are also contemplated.

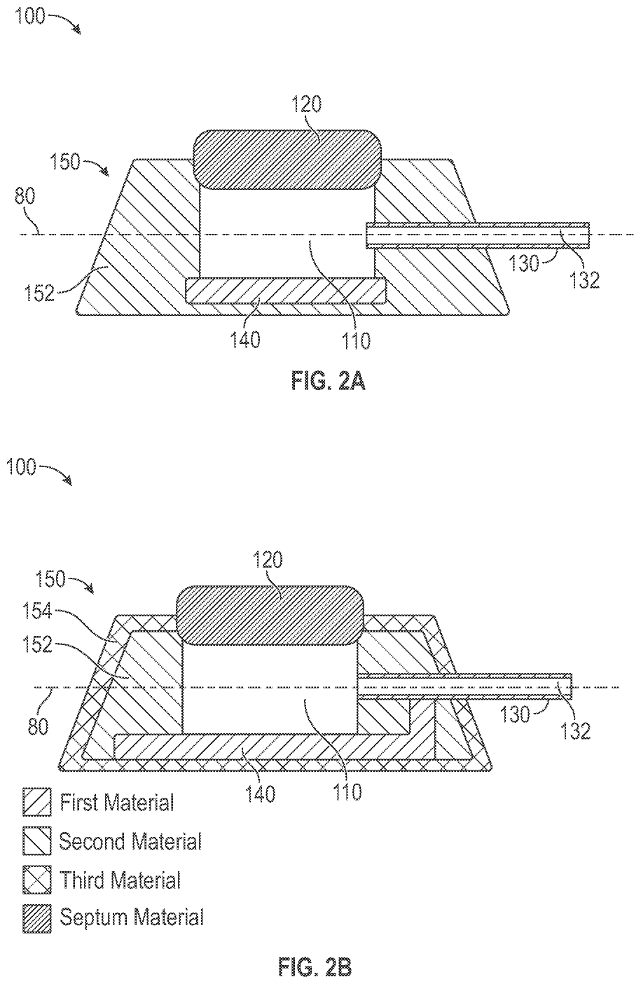

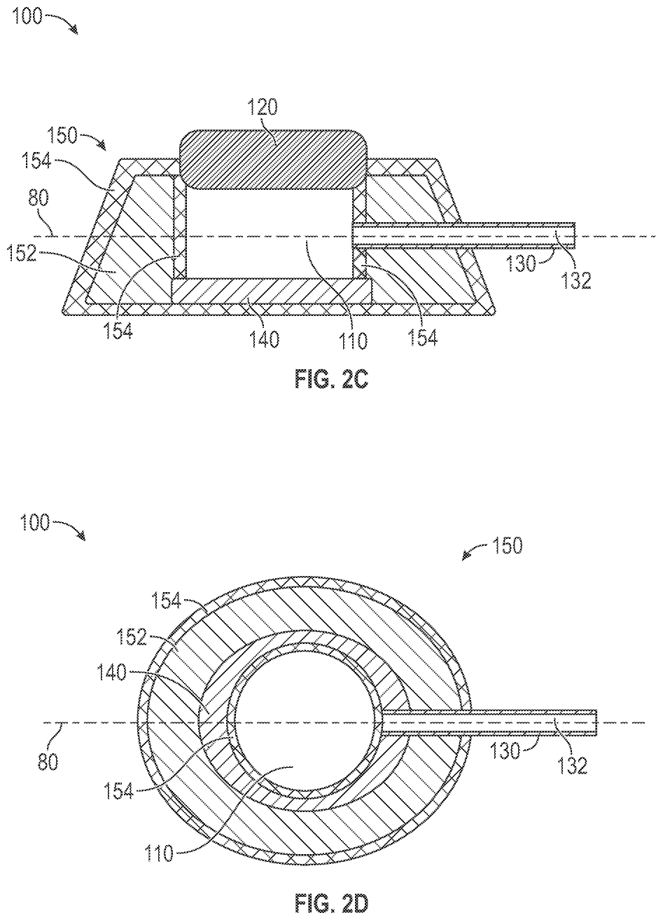

In an embodiment, as shown in FIG. 2A, the port body 150 can be formed of a second material. The second material can be different from the first material. In an embodiment, the second material can include a compliant material that can elastically deform. In an embodiment the second material can include a plastic, polymer, elastomer, organic or synthetic rubber, silicone, or the like. In an embodiment, the second material can include a second durometer, the second durometer being less than the first durometer of the first material. In an embodiment, the second durometer can be less than a Shore D 70 durometer. In an embodiment, the septum 120 can be formed of the same material as the second material. In an embodiment, the septum 120 can be formed of a different material from the first material.

In an embodiment, as shown in FIGS. 2A-2D, the port 100 can further include a needle-impenetrable base plate 140 disposed below the reservoir 110 and extending over at least a portion of a lower surface of the reservoir 110. FIG. 0.2D shows a plan view of the port 100 including a base plate 140 extending below at least a portion of the reservoir 110. In an embodiment, the base plate 140 can define the lower surface of the reservoir 110. In an embodiment, the base plate 140 can define a horizontal diameter or a horizontal surface area. The horizontal diameter or a horizontal surface area can be the same as or greater than a horizontal diameter or a horizontal surface area of the reservoir 110.

In an embodiment, the base plate 140 can be formed of a material that can resist penetration from a needle impinging thereon. In an embodiment, the base plate 140 can be formed of a substantially rigid material such as a plastic, polymer, metal, alloy, or composite, e.g. the first material, or the same material as the port stem 130 is formed of. In an embodiment, the base plate 130 can be formed of an elastically deformable, or plastically deformable (i.e. malleable), material that is configured to resist penetration by a needle impinging thereon, i.e. a flexible needle-impenetrable material. Exemplary materials can include plastics, polymers, metals, alloys, composites, KEVLAR R, or the like. Advantageously, the base plate 130 formed of a flexible needle-impenetrable material can prevent the needle from penetrating a floor of the reservoir 110 as well as allow the port 100 to collapse along a horizontal axis, e.g. longitudinal or lateral axes.

In an embodiment, as shown in FIG. 2B, the stem 130 and the base plate 140 can be formed from the same material and can be formed integrally as a single unitary piece. In an embodiment, as shown in FIG. 2A, the stem 130 and the base plate 140 can be formed as separate structures. In an embodiment, as shown in FIGS. 1 and 2D, the port 100 can define a substantially radially symmetrical shape extending about the central transvers axis. In an embodiment, the port 100 can define a substantially circular, elliptical, triangular, square, or rectangular shaped footprint, or plan view. However, it will be appreciated that other shapes are also contemplated.

In an embodiment, as shown in FIG. 2B, the port 100 can include a third material. The third material can include an elastically or plastically deformable material, including a plastic, polymer, elastomer, composite, organic or synthetic rubber, silicone material, or the like. In an embodiment, the third material can define a third durometer that is greater than a second durometer of the second material. In an embodiment, the third durometer can be the same as, or less than, the first durometer of the first material.

In an embodiment, the port body 150 can be formed of the third material and can define the reservoir 110. In an embodiment, as shown in FIG. 2B, the port body 150 can include the second material 152 and form the reservoir 110 and include the third material 154 disposed on an outer surface thereof to form a portion of the outer surface of the port body 150. In an embodiment, the third material 154 can extend over an entire outer surface of the port body 150 to form a “shell” surrounding the portions of the port body 150 formed of the second material 152. In an embodiment, as shown in FIG. 2C, a wall of the reservoir 110 can include the third material 154 disposed thereon.

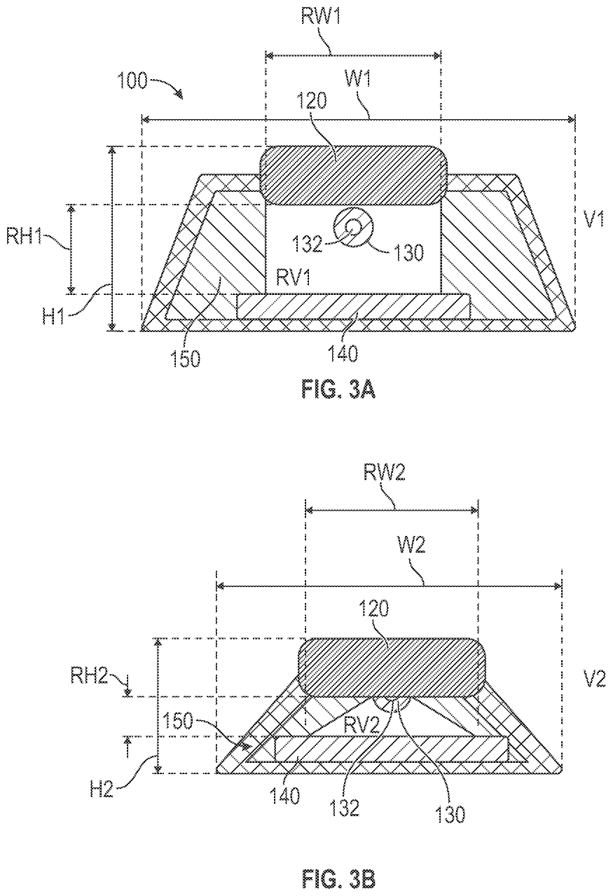

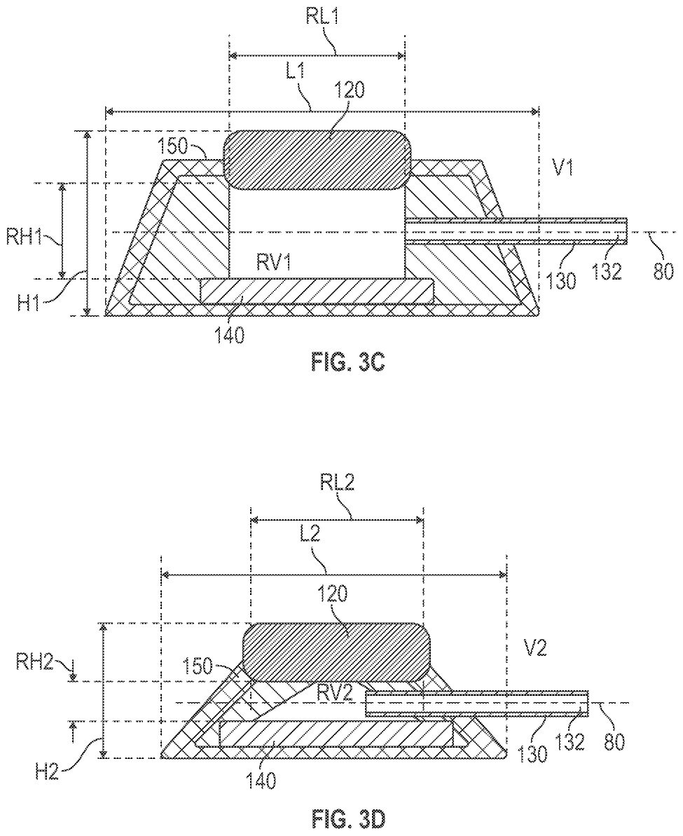

In an embodiment, as shown in FIGS. 3A-3D, the port 100 can be configured to transition between an expanded configuration (FIGS. 3A, 3C) and a collapsed configuration (FIGS. 3B, 3D). In an embodiment, in the expanded configuration the port body 150 can define a first port height (H1), a first port width (W1), or a first port length (L1), extending along the transverse, lateral, and longitudinal axes respectively. However, it will be appreciated that the height, width, or length can each extend along different axes. In an embodiment, the port body 150 can define a first port volume (V1).

In an embodiment, in the collapsed configuration the port body 150 can define a second port height (H2), a second port width (W2), a second port length (L2), or a second port volume (V2). In an embodiment, the second port height (H2) can be less than a first port height (H1). In an embodiment, the second port width (W2) can be less than a first port width (W1). In an embodiment, the second port length (L2) can be less than a first port length (L1). In an embodiment, the second port volume (V2) can be less than a first port volume (V1).

In an embodiment, in the expanded configuration the reservoir can define a first reservoir height (RH1), a first reservoir width (RW1), or a first reservoir length (RL1), extending along the transverse, lateral, and longitudinal axes respectively. However, it will be appreciated that the height, width, or length can each extend along different axes. In an embodiment, the reservoir 110 can define a first reservoir volume (RV1).

In an embodiment, in the collapsed configuration the reservoir 110 can define a second reservoir height (RH2), a second reservoir width (RW2), a second reservoir length (RL2), or a second reservoir volume (RV2). In an embodiment, the second reservoir height (RH2) can be less than a first reservoir height (RH1). In an embodiment, the second reservoir width (RW2) can be less than a first reservoir width (RW1). In an embodiment, the second reservoir length (RL2) can be less than a first reservoir length (RL1). In an embodiment, the second reservoir volume (RV2) can be less than a first reservoir volume (RV1). In an embodiment, the second reservoir volume (RV2) of the reservoir 110 can define a zero volume, or a de minimis volume.

In an embodiment, the port 100 can be biased towards the expanded configuration (FIGS. 3A, 3C). In the expanded configuration the port 100 can be configured to receive a needle extending percutaneously to traverse the septum 120 and access the reservoir 110 and disposed a fluid therein. The fluid can then pass through the stem lumen 132 and into the catheter lumen 92 to the vasculature of the patient.

In an embodiment, the port 100 can be elastically deformed from the expanded configuration to the collapsed configuration. In the collapsed configuration the port 100 can define a smaller dimension (e.g. height, width, length) or a smaller overall volume to facilitate placing the port subcutaneously. Advantageously, the collapsed configuration can require a smaller incision site to dispose the port subcutaneously, leading to less stitches or no stitches at all in order to close the incision site, reducing scaring, improving patient recovery times, and/or improving aesthetics. Once placed subcutaneously, the port 100 can resume the expanded configuration ready for use.

In an embodiment, the reservoir 110 can elastically deform between the expanded configuration and the collapsed configuration to allow the port 100 to elastically deform between the expanded configuration and the collapsed configuration. In an embodiment, the port 100 can elastically deform along a first axis and elastically expand along a second axis, extending at an angle to the first axis. As such, the port body 150 can elastically deform to reduce a cross-sectional area in a first plane and fit through a smaller incision site than would otherwise be required in the expanded configuration. For example, the port body 150 can be configured to elastically deform along one of the transverse or lateral axes and can elastically expand along the longitudinal axes to allow a cross-sectional area of the port body 150, extending along a laterally vertical plane, to reduce. As such, the port 100 can fit through a smaller insertion site than would be required in the expanded configuration.

In an embodiment, the port 100 can be biased to the expanded configuration and can include a vacuum disposed within the reservoir 110 and configured to maintain the port 100 in the collapsed configuration. The port 100 can then be inserted through the insertion site and disposed subcutaneously. Once placed subcutaneously, the vacuum can be released from the reservoir 110 and the port 100 can transition from the collapsed configuration to the expanded configuration. In an embodiment, the vacuum can be maintained within the reservoir 110 by sealing the stem lumen 132. Once the port 100 is placed subcutaneously, the seal in the stem lumen 132 can be broken, releasing the vacuum. In an embodiment, the vacuum in the reservoir 110 can be release by accessing the port percutaneously with a needle, through the needle penetrable septum 120. Advantageously, the vacuum within the reservoir 110 can draw a fluid through the access needle and into the reservoir 110 as the port 100 transitions from the collapsed configuration to the expanded configuration.

In an embodiment, the port 100 can be biased towards the collapsed configuration. In an embodiment, the port 100 can maintain the collapsed configuration during subcutaneous placement and does not require confinement or constraint in order to maintain the collapsed configuration during placement. The port 100 can then be transitioned from the collapsed configuration to the expanded configuration once placed subcutaneously.

In an embodiment, the port 100, biased towards the collapsed configuration, can be disposed subcutaneously and can be accessed by a needle extending percutaneously. The needle can provide a pressurized fluid to the reservoir 110 of the port 100. The reservoir 110 can be configured to transition to the expanded configuration as the fluid is introduced to the port 100. Once the fluid flow has ceased and the fluid has pass through the stem 130 to the catheter 90, the port 100 can transition from the expanded configuration to the collapsed configuration. Advantageously, the port 100 biased towards the collapsed configuration can require a smaller insertion site compared to the port 100 in the expanded configuration. Further, the port 100 can remain in the collapsed configuration until accessed by a needle. This can provide a lower profile, reducing scaring, reducing skin stretching, and improving aesthetics, in between access events. In an embodiment, the port 100 can be bistable in both the expanded configuration and the collapsed configuration.

Advantageously, the port body 150 formed of the compliant material(s), i.e. the second material and/or the third material, and elastically deformable between the expanded configuration and the collapsed configuration, can improve patient comfort when placed subcutaneously. Advantageously, the embodiments of the port 100 can provide a lower risk of erosion.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A subcutaneous access port, comprising:
a port stem formed of a first material, being a rigid material and having a first durometer; and
a body defining a reservoir in fluid communication with the port stem, and formed of a second material, being a flexible material and having a second durometer, the body transitionable between an expanded configuration and a collapsed configuration, the collapsed configuration defining a smaller outer profile of the port.

2. The subcutaneous access port according to claim 1, wherein the first material includes one of a plastic, polymer, metal, alloy, or composite.

3. The subcutaneous access port according to claim 1, wherein the second material includes one of a plastic, polymer, elastomer, synthetic rubber, organic rubber, silicone rubber, or composite.

4. The subcutaneous access port according to claim 1, wherein the port body in the expanded configuration defines one of a first port height, a first port width, or a first port length, and wherein the port body in the collapsed configuration defines one of a second port height, a second port width, or a second port length.

5. The subcutaneous access port according to claim 4, wherein one of the second port height is less than the first port height, the second port width is less than the first port width, or the second port length is less than the first port length.

6. The subcutaneous access port according to claim 1, wherein the port body in the expanded configuration defines a first port volume, and the port body in the collapsed configuration defines a second port volume, the second port volume being less than the first port volume.

7. The subcutaneous access port according to claim 1, wherein the reservoir in the expanded configuration defines one of a first reservoir height, a first reservoir width, or a first reservoir length, and wherein the reservoir in the collapsed configuration defines one of a second reservoir height, a second reservoir width, or a second reservoir length.

8. The subcutaneous access port according to claim 7, wherein one of the second reservoir height is less than the first reservoir height, the second reservoir width is less than the first reservoir width, or the second reservoir length is less than the first reservoir length.

9. The subcutaneous access port according to claim 1, wherein the reservoir in the expanded configuration defines a first reservoir volume, and the reservoir in the collapsed configuration defines a second reservoir volume, the second volume being less than the first volume.

10. The subcutaneous access port according to claim 1, further including a needle penetrable septum disposed over the reservoir and configured to provide percutaneous access thereto by a needle.

11. The subcutaneous access port according to claim 10, wherein the needle penetrable septum is formed of either the second material or a silicone rubber.

12. The subcutaneous access port according to claim 1, wherein the first durometer of the first material is larger than the second durometer of the second material.

13. The subcutaneous access port according to claim 1, wherein the first material is a rigid material and is substantially resistant to flexible deformation, and wherein the second material is an elastically deformable material.

14. The subcutaneous access port according to claim 1, wherein the body is elastically deformable from the expanded configuration to the collapsed configuration.

15. The subcutaneous access port according to claim 1, wherein the body is elastically deformable from the collapsed configuration to the expanded configuration.

16. The subcutaneous access port according to claim 1, further including a third material, being elastically deformable and including a third durometer greater than the second durometer and less than the first durometer.

17. The subcutaneous access port according to claim 16, wherein the third material is disposed on an outer surface of the body.

18. The subcutaneous access port according to claim 16, wherein the third material is disposed on a wall of the reservoir.

19. The subcutaneous access port according to claim 1, further including a base plate formed of one of the first material or a flexible needle impenetrable material.

20. The subcutaneous access port according to claim 19, wherein the base plate and the stem are formed integrally as a single unitary piece.

21-43. (canceled)
